(19) United States
(12) Patent Application Publication
(10) Pub. No.: US 2024/0318207 A1
(43) Pub. Date: Sep. 26, 2024

(54) INCREASED ETHANOL PRODUCTION BY OVER-EXPRESSION OF KGD2 IN YEAST

(71) Applicant: DANISCO US INC., Palo Alto, CA (US)

(72) Inventors: Min Qi, Hockessin, DE (US); Yehong Jamie Wang, Wilmington, DE (US); Quinn Qun Zhu, West Chester, PA (US)

(21) Appl. No.: 18/290,026

(22) PCT Filed: May 10, 2022
(86) PCT No.: PCT/US2022/028530

§ 371 Date: Nov. 9, 2023

Related U.S. Application Data
(60) Provisional application No. 63/186,332, filed on May 10, 2021.

(51) Int. Cl. C12P 7/06; C12N 1/18; C12N 9/02; C12N 15/81; C12R 1/865

(52) U.S. Cl. CPC C12P 7/06; C12N 1/185; C12N 9/0008; C12N 15/81; C12R 2001/865; C12Y 102/04002

(57) ABSTRACT

Described are compositions and methods relating to modified yeast cells that over-express α-ketoglutarate dehydrogenase (K.GD2). Die modified yeast cells produce increased amounts of ethanol compared to otherwise identical parental yeast cells. Such yeast cells are particularly useful for large-scale ethanol production from starch substrates.

### CROSS REFERENCE

This application claims the benefit of U.S. Provisional Application No. 63/186,332, filed May 10, 2021, which is incorporated by reference in its entirety.

### INCORPORATION BY REFERENCE OF THE SEQUENCE LISTING

The present application is being filed along with a Sequence Listing in electronic format. The Sequence Listing is provided as a file entitled NB41694WOPCTSeqList.txt, created May 9, 2022, which is 14,936 bytes in size. The information in the electronic format of the Sequence Listing is incorporated by reference in its entirety.

### TECHNICAL FIELD

The present compositions and methods relate to modified yeast that over-expresses α-ketoglutarate dehydrogenase (KGD2). The yeast produces an increased amount of ethanol compared to otherwise identical parental cells. Such yeast is particularly useful for large-scale ethanol production from starch substrates.

### BACKGROUND

First-generation yeast-based ethanol production converts sugars into fuel ethanol. The annual fuel ethanol production by yeast is about 90 billion liters worldwide (Gombert, A. K. and van Maris. A. J. (2015) Curr. Opin. Biotechnol. 33:81-86). It is estimated that about 70% of the cost of ethanol production is the feedstock. Since the production volume is so large, even small yield improvements have massive economic impact across the industry. Accordingly, the need exists for robust yeast that make more ethanol compared to both conventional and engineered yeast.

### SUMMARY

The present compositions and methods relate to modified yeast that over-expresses α-ketoglutarate dehydrogenase (KGD2). Aspects and embodiments of the compositions and methods are described in the following, independently-numbered, paragraphs.

1. In one aspect, modified yeast cells derived from parental yeast cells are provided, the modified cells comprising a genetic alteration that causes the modified cells to produce an increased amount of α-ketoglutarate dehydrogenase (KGD2) polypeptides compared to the parental cells, wherein the modified cells produce during fermentation an increased amount of ethanol compared to the amount of ethanol produced by otherwise identical parental yeast cells.

2. In some embodiments of the modified cells of paragraph 1, the genetic alteration comprises the introduction into the parental cells of a nucleic acid capable of directing the expression of a KGD2 polypeptide to a level above that of the parental cell grown under equivalent conditions.

3. In some embodiments of the modified cells of paragraph 2, the genetic alteration comprises the introduction of an expression cassette for expressing a KGD2 polypeptide.

4. In some embodiments of the modified cells of paragraph 3, the expression cassette comprises an exogenous KGD2 gene.

5. In some embodiments of the modified cells of paragraph 2, the nucleic acid comprises a promoter that results in increased expression of KGD2 polypeptides late in fermentation.

6. In some embodiments of the modified cells of paragraph 2, the nucleic acid comprises the ADR1 promoter operably linked to the coding sequence for the KGD2 polypeptide.

7. In some embodiments of the modified cells of any of paragraphs 1-6, the amount of increase in the expression of a KGD2 polypeptide is at least 20%, at least 30%, at least 40%, at least 50%, at least 70%, at least 100%, at least 150%, at least 200%, or at least 500% or more, compared to the level expression in the parental cells grown under equivalent conditions.

8. In some embodiments of the modified cells of any of paragraphs 1-6, the increase in the amount of KGD2 mRNA produced by the modified cells is at least 2-fold, at least 5-fold, at least 10-fold, at least 20-fold, at least 50-fold, at least 100-fold or more, compared to the amount of KGD2 mRNA produced by the parental cells grown under equivalent conditions.

9. In some embodiments of the modified cells of any of paragraphs 1-8, the cells further comprise a genetic alteration that causes the modified cells to produce an increased amount of transcriptional regulator MIG3 polypeptides compared to the parental cells.

10. In some embodiments of the modified cells of any of paragraphs 1-9, the cells further comprise an exogenous gene encoding a carbohydrate processing enzyme.

11. In some embodiments, the modified cells of any of paragraphs 1-10 further comprise a PKL pathway.

12. In some embodiments, the modified cells of any of paragraphs 1-11 further comprise an alteration in the glycerol pathway and/or the acetyl-CoA pathway.

13. In some embodiments, the modified cells of any of paragraphs 1-12 further comprise an alternative pathway for making ethanol.

14. In some embodiments of the modified cells of any of paragraphs 1-13, the cells are of a Saccharomyces spp.

15. In another aspect, a method for increased production of alcohol from yeast cells grown on a carbohydrate substrate is provided, comprising: introducing into parental yeast cells a genetic alteration that increases the production of KGD2 polypeptides compared to the amount produced in otherwise identical parental cells.

16. In some embodiments of the method of paragraph 15, the modified cells having the introduced genetic alteration are the modified cells are the cells of any of paragraphs 1-14.

17. In some embodiments of the method of paragraph 15 or 16, the increased production of alcohol is at least 0.5% under equivalent fermentation conditions.

18. In some embodiments of the method of any of paragraphs 15-17, the increase in production of KGD2 is an increase of at least 20%, at least 30%, at least 40%, at least 50%, at least 70%, at least 100%, at least 150%, at least 200%, or at least 500% or more compared to the amount of KGD2 produced by otherwise identical parental cells grown under equivalent conditions.

19. In some embodiments of the method of any of paragraphs 15-18, the increase in the amount of KGD2 mRNA produced by the modified cells is at least 2-fold, at least 5-fold, at least 10-fold, at least 20-fold, at least 50-fold, at least 100-fold or more compared to the amount of KGD2 mRNA produced by otherwise identical parental cells grown under equivalent conditions.

These and other aspects and embodiments of present modified cells and methods will be apparent from the description, including any accompanying Drawings/Figures.

### DETAILED DESCRIPTION

### I. Definitions

Prior to describing the present yeast and methods in detail, the following terms are defined for clarity. Terms not defined should be accorded their ordinary meanings as used in the relevant art.

As used herein, the term “alcohol” refers to an organic compound in which a hydroxyl functional group (—OH) is bound to a saturated carbon atom.

As used herein, the terms “yeast cells,” “yeast strains,” or simply “yeast” refer to organisms from the phyla Ascomycota and Basidiomycota. Exemplary yeast is budding yeast from the order Saccharomycetales. Particular examples of yeast are Saccharomyces spp., including but not limited to S. cerevisiae. Yeast include organisms used for the production of fuel alcohol as well as organisms used for the production of potable alcohol, including specialty and proprietary yeast strains used to make distinctive-tasting beers, wines, and other fermented beverages.

As used herein, the phrase “engineered yeast cells,” “variant yeast cells,” “modified yeast cells,” or similar phrases, refer to yeast that include genetic modifications and characteristics described herein. Variant/modified yeast do not include naturally occurring yeast.

As used herein, the terms “polypeptide” and “protein” (and their respective plural forms) are used interchangeably to refer to polymers of any length comprising amino acid residues linked by peptide bonds. The conventional one-letter or three-letter codes for amino acid residues are used herein and all sequence are presented from an N-terminal to C-terminal direction. The polymer can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

As used herein, functionally and/or structurally similar proteins are considered to be “related proteins,” or “homologs.” Such proteins can be derived from organisms of different genera and/or species, or different classes of organisms (e.g., bacteria and fungi), or artificially designed. Related proteins also encompass homologs determined by primary sequence analysis, determined by secondary or tertiary structure analysis, or determined by immunological cross-reactivity, or determined by their functions.

As used herein, the term “homologous protein” refers to a protein that has similar activity and/or structure to a reference protein. It is not intended that homologs necessarily be evolutionarily related. Thus, it is intended that the term encompass the same, similar, or corresponding enzyme(s) (i.e., in terms of structure and function) obtained from different organisms. In some embodiments, it is desirable to identify a homolog that has a quaternary, tertiary and/or primary structure similar to the reference protein. In some embodiments, homologous proteins induce similar immunological response(s) as a reference protein. In some embodiments, homologous proteins are engineered to produce enzymes with desired activity(ies).

The degree of homology between sequences can be determined using any suitable method known in the art (see. e.g., Smith and Waterman (1981) Adv. Appl. Math. 2:482; Needleman and Wunsch (1970) J. Mol. Biol., 48:443; Pearson and Lipman (1988) Proc. Natl. Acad. Sci. USA 85:2444; programs such as GAP, BESTFIT, FASTA, and TFASTA in the Wisconsin Genetics Software Package (Genetics Computer Group, Madison, WI); and Devereux et al. (1984) Nucleic Acids Res. 12:387-95).

For example, PILEUP is a useful program to determine sequence homology levels. PILEUP creates a multiple sequence alignment from a group of related sequences using progressive, pair-wise alignments. It can also plot a tree showing the clustering relationships used to create the alignment. PILEUP uses a simplification of the progressive alignment method of Feng and Doolittle, (Feng and Doolittle (1987) J. Mol. Evol. 35:351-60). The method is similar to that described by Higgins and Sharp ((1989) CABIOS 5:151-53). Useful PILEUP parameters including a default gap weight of 3.00, a default gap length weight of 0.10, and weighted end gaps. Another example of a useful algorithm is the BLAST algorithm, described by Altschul et al. ((1990) J. Mol. Biol. 215:403-10) and Karlin et al. ((1993) Proc. Nat. Acad. Sci. USA 90:5873-87). One particularly useful BLAST program is the WU-BLAST-2 program (see, e.g., Altschul et al. (1996) Meth. Enzymol. 266:460-80). Parameters “W,” “T,” and “X” determine the sensitivity and speed of the alignment. The BLAST program uses as defaults a word-length (W) of 11, the BLOSUM62 scoring matrix (see, e.g., Henikoff and Henikoff (1989) Proc. Natl. Acad. Sci. USA 89:10915) alignments (B) of 50, expectation (E) of 10, M′5, N′-4, and a comparison of both strands.

As used herein, the phrases “substantially similar” and “substantially identical,” in the context of at least two nucleic acids or polypeptides, typically means that a polynucleotide or polypeptide comprises a sequence that has at least about 70% identity, at least about 75% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or even at least about 99% identity, or more, compared to the reference (i.e., wild-type) sequence. Percent sequence identity is calculated using CLUSTAL W algorithm with default parameters. See Thompson et al. (1994) Nucleic Acids Res. 22:4673-4680. Default parameters for the CLUSTAL W algorithm are:

| | |
|---|---|
| Gap opening penalty: | 10.0 |
| Gap extension penalty: | 0.05 |
| Protein weight matrix: | BLOSUM series |
| DNA weight matrix: | IUB |
| Delay divergent sequences %: | 40 |
| Gap separation distance: | 8 |
| DNA transitions weight: | 0.50 |
| List hydrophilic residues: | GPSNDQEKR |
| Use negative matrix: | OFF |
| Toggle Residue specific penalties: | ON |
| Toggle hydrophilic penalties: | ON |
| Toggle end gap separation penalty | OFF |

Another indication that two polypeptides are substantially identical is that the first polypeptide is immunologically cross-reactive with the second polypeptide. Typically, polypeptides that differ by conservative amino acid substitutions are immunologically cross-reactive. Thus, a polypeptide is substantially identical to a second polypeptide, for example, where the two peptides differ only by a conservative substitution. Another indication that two nucleic acid sequences are substantially identical is that the two molecules hybridize to each other under stringent conditions (e.g., within a range of medium to high stringency).

As used herein, the term “gene” is synonymous with the term “allele” in referring to a nucleic acid that encodes and directs the expression of a protein or RNA. Vegetative forms of filamentous fingi are generally haploid, therefore a single copy of a specified gene (i.e., a single allele) is sufficient to confer a specified phenotype. The term “allele” is generally preferred when an organism contains more than one similar genes, in which case each different similar gene is referred to as a distinct “allele.”

As used herein, “constitutive” expression refers to the production of a polypeptide encoded by a particular gene under essentially all typical growth conditions, as opposed to “conditional” expression, which requires the presence of a particular substrate, temperature, or the like to induce or activate expression.

As used herein, the term “expressing a polypeptide” and similar terms refers to the cellular process of producing a polypeptide using the translation machinery (e.g., ribosomes) of the cell.

As used herein, “over-expressing a polypeptide,” “increasing the expression of a polypeptide,” and similar terms, refer to expressing a polypeptide at higher-than-normal levels compared to those observed with parental or “wild-type cells that do not include a specified genetic modification.

As used herein, an “expression cassette” refers to a DNA fragment that includes a promoter, and amino acid coding region and a terminator (i.e., promoter::amino acid coding region::terminator) and other nucleic acid sequence needed to allow the encoded polypeptide to be produced in a cell. Expression cassettes can be exogenous (i.e., introduced into a cell) or endogenous (i.e., extant in a cell).

As used herein, the terms “fused” and “fusion” with respect to two DNA fragments, such as a promoter and the coding region of a polypeptide refer to a physical linkage causing the two DNA fragments to become a single molecule.

As used herein, the terms “wild-type” and “native” are used interchangeably and refer to genes, proteins or strains found in nature, or that are not intentionally modified for the advantage of the presently described yeast.

As used herein, the term “protein of interest” refers to a polypeptide that is desired to be expressed in modified yeast. Such a protein can be an enzyme, a substrate-binding protein, a surface-active protein, a structural protein, a selectable marker, or the like, and can be expressed. The protein of interest is encoded by an endogenous gene or a heterologous gene (i.e., gene of interest”) relative to the parental strain. The protein of interest can be expressed intracellularly or as a secreted protein or displayed on cell the surface.

As used herein, “disruption of a gene” refers broadly to any genetic or chemical manipulation, i.e., mutation, that substantially prevents a cell from producing a function gene product, e.g., a protein, in a host cell. Exemplary methods of disruption include complete or partial deletion of any portion of a gene, including a polypeptide-coding sequence, a promoter, an enhancer, or another regulatory element, or mutagenesis of the same, where mutagenesis encompasses substitutions, insertions, deletions, inversions, and combinations and variations, thereof, any of which mutations substantially prevent the production of a function gene product. A gene can also be disrupted using CRISPR, RNAi, antisense, or any other method that abolishes gene expression. A gene can be disrupted by deletion or genetic manipulation of non-adjacent control elements. As used herein, “deletion of a gene,” refers to its removal from the genome of a host cell. Where a gene includes control elements (e.g., enhancer elements) that are not located immediately adjacent to the coding sequence of a gene, deletion of a gene refers to the deletion of the coding sequence, and optionally adjacent enhancer elements, including but not limited to, for example, promoter and/or terminator sequences, but does not require the deletion of non-adjacent control elements. Deletion of a gene also refers to the deletion a part of the coding sequence, or a part of promoter immediately or not immediately adjacent to the coding sequence, where there is no functional activity of the interested gene existed in the engineered cell.

As used herein, the terms “genetic manipulation,” “genetic alteration”, “genetic engineering”, and similar terms are used interchangeably and refer to the alteration/change of a nucleic acid sequence. The alteration can include but is not limited to a substitution, deletion, insertion or chemical modification of at least one nucleic acid in the nucleic acid sequence.

As used herein, a “functional polypeptide/protein” is a protein that possesses an activity, such as an enzymatic activity, a binding activity, a surface-active property, or the like, and which has not been mutagenized, truncated, or otherwise modified to abolish or reduce that activity. Functional polypeptides can be thermostable or thermolabile, as specified.

As used herein, the terms “fused protein” and “fusion protein” with respect to two polypeptides, such as two different enzymes physically linked together with or without a linker(s) causing the two polypeptides to become a single molecule.

As used herein, “a functional gene” is a gene capable of being used by cellular components to produce an active gene product, typically a protein. Functional genes are the antithesis of disrupted genes, which are modified such that they cannot be used by cellular components to produce an active gene product, or have a reduced ability to be used by cellular components to produce an active gene product.

As used herein, yeast cells have been “modified to prevent the production of a specified protein” if they have been genetically or chemically altered to prevent the production of a functional protein/polypeptide that exhibits an activity characteristic of the wild-type protein. Such modifications include, but are not limited to, deletion or disruption of the gene encoding the protein (as described, herein), modification of the gene such that the encoded polypeptide lacks the aforementioned activity, modification of the gene to affect post-translational processing or stability, and combinations, thereof.

As used herein, “attenuation of a pathway” or “attenuation of the flux through a pathway,” i.e., a biochemical pathway, refers broadly to any genetic or chemical manipulation that reduces or completely stops the flux of biochemical substrates or intermediates through a metabolic pathway. Attenuation of a pathway may be achieved by a variety of well-known methods. Such methods include but are not limited to: complete or partial deletion of one or more genes, replacing wild-type alleles of these genes with mutant forms encoding enzymes with reduced catalytic activity or increased Km values, modifying the promoters or other regulatory elements that control the expression of one or more genes, engineering the enzymes or the mRNA encoding these enzymes for a decreased stability, misdirecting enzymes to cellular compartments where they are less likely to interact with substrate and intermediates, the use of interfering RNA, and the like.

As used herein, “aerobic fermentation” refers to growth and production process in the presence of oxygen.

As used herein, “anaerobic fermentation” refers to growth and production in the absence of oxygen.

As used herein, the expression “end of fermentation” refers to the stage of fermentation when the economic advantage of continuing fermentation to produce a small amount of additional alcohol is exceeded by the cost of continuing fermentation in terms of fixed and variable costs. In a more general sense, “end of fermentation” refers to the point where a fermentation will no longer produce a significant amount of additional alcohol. i.e., no more than about 1% additional alcohol.

As used herein, the expression “carbon flux” refers to the rate of turnover of carbon molecules through a metabolic pathway. Carbon flux is regulated by enzymes involved in metabolic pathways, such as the pathway for glucose metabolism and the pathway for maltose metabolism.

As used herein, the singular articles “a,” “an” and “the” encompass the plural referents unless the context clearly dictates otherwise. All references cited herein are hereby incorporated by reference in their entirety. The following abbreviations/acronyms have the following meanings unless otherwise specified:

following meanings unless otherwise specified:

| | |
|---|---|
| ° C. | degrees Centigrade |
| AA | α-amylase |
| AADH | acetaldehyde dehydrogenases |
| TCA | tricarboxylic acid |
| bp | base pairs |
| DNA | deoxyribonucleic acid |
| ds or DS | dry solids |
| EC | enzyme commission |
| EtOH | ethanol |
| g or gm | gram |
| g/L | grams per liter |
| GA | glucoamylase |
| H₂O | water |
| HPLC | high performance liquid chromatography |
| hr or h | hour |
| KGD2 | α-ketoglutarate dehydrogenase |
| kg | kilogram |
| M | molar |
| mg | milligram |
| min | minute |
| mL or ml | milliliter |
| mM | millimolar |
| mRNA | messenger RNA |
| N | normal |
| nm | nanometer |
| PCR | polymerase chain reaction |
| PKL | phosphoketolase |
| ppm | parts per million |
| PTA | phosphotransacetylase |
| Δ | relating to a deletion |
| μg | microgram |
| μL and μl | microliter |
| μM | micromolar |

### II. Modified Yeast Cells Having Increased KGD2 Expression

The present compositions and methods relate to modified yeast cells having a genetic alteration that causes the cells to produce an increased amount of α-ketoglutarate dehydrogenase (KGD2) polypeptides compared to otherwise identical parental cells, wherein the modified cells produce during fermentation an increased amount of ethanol compared to the amount of ethanol produced by the otherwise identical parental cells under equivalent fermentation conditions.

KGD2 is a component of the mitochondrial α-ketoglutarate dehydrogenase complex, which catalyzes the oxidative decarboxylation of α-ketoglutarate to succinyl-CoA in the tricarboxylic acid cycle (TCA) cycle. KGD2 is essential for aerobic growth of yeast, but it's role in anaerobic fermentation is heretofore unknown.

Based on analyses of metabolic pathways and previous experience with yeast having engineered metabolic pathways, it was postulated that over-expression of KGD2 late in fermentation could lead to increased ethanol production under anaerobic conditions, such as those found in commercial ethanol production facilities. Indeed, as evidenced by the appended Examples, late stage over-expression of KGD2 in a commercially available yeast resulted in significantly increased ethanol production

The amino acid sequence of the exemplified S. cerevisiae KGD2 polypeptide is shown, below, as SEQ ID NO: 1:

MLSRATRTAAAKSLVKSKVARNVMAASFVKRHASTSLFKQANKVE
SLGSIYLSGKKISVAANPFSITSNRFKSTSIEVPPMAESLTEGSL
KEYTKNVGDFIKEDELLATIETDKIDIEVNSPVSGTVTKLNFKPE
DTVTVGEELAQVEPGEAPAEGSGESKPEPTEQAEPSQGVAARENS
SEETASKKEAAPKKEAAPKKEVTEPKKADQPKKTVSKAQEPPVAS
NSFTPFPRTETRVKMNRMRLRIAERLKESQNTAASLTTFNEVDMS
ALMEMRKLYKDEIIKKTGTKFGFMGLFSKACTLAAKDIPAVNGAI
EGDQIVYRDYTDISVAVATPKGLVTPVVRNAESLSVLDIENEIVR
LSHKARDGKLTLEDMTGGTFTISNGGVFGSLYGTPIINSPQTAVL
GLHGVKERPVTVNGQIVSRPMMYLALTYDHRLLDGREAVTFLKTV
KELIEDPRKMLLW

The NCBI database includes entries for polypeptides from numerous organisms having varying degrees of identity to SEQ ID NO: 1. These polypeptides are expected to function similarly when introduced into yeast, particularly in view of the fact that the TCA cycle is essentially ubiquitous in nature.

In particular embodiments of the present compositions and methods, the amino acid sequence of the KGD2 polypeptide that is over-expressed in modified yeast cells has at least about 60%, at least about 65%, at least about 70%, at least about 75%, at least about 80%, at least about 85%, at least about 90%, at least about 95%, at least about 97%, at least about 98% or even at least about 99% to SEQ ID NO: 1.

Natural variations in the amino acid sequence are not expected to affect function. Moreover, over-expression of functionally and/or structurally similar proteins, homologous proteins and/or substantially similar or identical proteins, is expected to produce similar beneficial results.

In some embodiments, the increase in the amount of KGD2 polypeptides produced by the modified cells is an increase of at least 20%, at least 30%, at least 40%, at least 50%, at least 70%, at least 100%, at least 150%, at least 200%, at least 500%, at least 1,000%, at least 2,000%, or more, compared to the amount of KGD2 polypeptides produced by otherwise identical parental cells grown under the same conditions.

In some embodiments, the increase in the amount of KGD2 mRNA produced by the modified cells is at least 2-fold, at least 5-fold, at least 10-fold, at least 20-fold, at least 50-fold, at least 100-fold or more, compared to the amount of KGD2 mRNA produced by otherwise identical parental cells grown under the same conditions.

In some embodiments, the increase in the strength of the promoter used to control expression of the KGD2 polypeptides produced by the modified cells is at least 2-fold, at least 5-fold, at least 10-fold, at least 20-fold, at least 50-fold, at least 100-fold or more, compared to strength of the native promoter controlling KGD2 expression, based on the amount of mRNA produced.

In some embodiments, the promoter directs maximum expression of KGD2 polypeptides late in fermentation, e.g., in the second half or last third or quarter of a typical industrial alcohol fermentation process, for example, a 48-hr fermentation process. In some embodiments, expression of KGD2 polypeptides is greater at 48 hr than at 24 hr. In some embodiments, expression of KGD2 polypeptides is greater at 48 hr than at 36 hr.

In some embodiments, the increase in ethanol production by the modified cells is an increase of at least about 0.5%, at least about 1.0%, at least about 1.5%, at least about 2.0%, at least 2.5% or more, compared to the amount of ethanol produced by otherwise identical cells grown under the same conditions.

Preferably, increased KGD2 expression is achieved by genetic manipulation using sequence-specific molecular biology techniques, as opposed to chemical mutagenesis, which is generally not targeted to specific nucleic acid sequences. However, chemical mutagenesis is not excluded as a method for making modified yeast cells.

In some embodiments, the present compositions and methods involve introducing into yeast cells a nucleic acid capable of directing the over-expression, or increased expression, of a KGD2 polypeptide. Particular methods include but are not limited to (i) introducing additional copies of an endogenous expression cassette for increased production of the polypeptide into a host cell, (ii) introducing an exogenous expression cassette(s) for increased production of polypeptide into a host cell, (iii) substituting an endogenous cassette with an exogenous expression cassette that allows the production of an increased amount of the polypeptide, (iv) modifying or replacing the promoter of an endogenous expression cassette to increase expression, and/or (v) modifying any aspect of the host cell to increase the half-life of the polypeptide in the host cell.

In some embodiments, the parental cell that is modified already includes a gene of interest, such as a gene encoding a selectable marker, carbohydrate-processing enzyme, or other polypeptide. In some embodiments, a gene of interest is subsequently introduced into the modified cells.

### III. Modified Yeast Cells Having Increased KGD2 and MIG3 Expression

Over-expression of KGD2 may advantageously be combined with over-expression of MIG3, a transcriptional regulator that reduces acetate and glycerol production.

In some embodiments, the increase in the amount of MIG3 polypeptides produced by the modified cells is an increase of at least about 20%, at least about 30%, at least about 40%, at least about 50%, at least about 70%, at least about 100%, at least about 150%, at least about 200%, at least about 500%, at least about 1,000%, at least about 2,000%, or more, compared to the amount of MIG3 polypeptides produced by otherwise identical parental cells grown under the same conditions.

In some embodiments, the increase in the amount of MIG3 mRNA produced by the modified cells is at least about 2-fold, at least about 5-fold, at least about 10-fold, at least about 20-fold, at least about 50-fold, at least about 100-fold or more, compared to the amount of MIG3 polypeptides produced by otherwise identical parental cells grown under the same conditions.

In some embodiments, the increase in the strength of the promoter used to control expression of the MIG3 polypeptides produced by the modified cells is at least about 2-fold, at least about 5-fold, at least about 10-fold, at least about 20-fold, at least about 50-fold, at least about 100-fold or more, compared to strength of the native promoter controlling MIG3 expression, based on the amount of mRNA produced. In some embodiments, the promoter is weaker that the EFB1 promoter. In particular embodiments, the promoter is the SU13 promoter.

In some embodiments, the decrease in acetate production by the modified cells is a decrease of at least about 5%, at least about 10%, at least about 15%, at least about 20%, at least about 25%, at least about 30%, at least about 35%, at least about 40%, or more, compared to the amount of acetate produced by otherwise identical parental cells grown under the same conditions.

In some embodiments, the reduction in glycerol in KGD2 and MIG3-expressing yeast is at least about 5%, at least about 8%, at least about 10%, at least about 12% or more, compared to the amount of acetate produced by otherwise identical parental cell.

Methods for introducing into yeast cells a nucleic acid capable of directing the over-expression, or increased expression, of a MIG3 polypeptide include those described, above for the over-expression of KGD2 polypeptides.

The amino acid sequence of the exemplified S. cerevisiae MIG3 polypeptide is shown, below, as SEQ ID NO: 5:

MNYLRDRFPPDNDQRPFRCEICSRGFHRLEHKKRHVRTHTGEKPH
KCTVQGCPKSFSRSDELKRHLRTHTKGVQRRRIKSKGSRKTVVNT
ATAAPTTFNENTGVSLTGIGQSKVPPILISVAQNCDDVNIRNTGN
NNGIVETQAPAILVPVINIPNDPHPIPSSLSTTSITSIASVYPST
SPFQYLKSGFPEDPASTPYVHSSGSSLALGELSSNSSIFSKSRRN
LAAMSGPDSLSSSKNQSSASLLSQTSHPSKSFSRPPTDLSPLRRI
MPSVNTGDMEISRTVSVSSSSSSLTSVTYDDTAAKDMGMGIFFDR
PPVTQKACRSNHKYKVNAVSRGRQHERAQFHISGDDEDSNVHRQE
SRASNTSPNVSLPPIKSILRQIDNFNSAPSYFSK

The NCBI database includes over 40 entries for S. cerevisiae MIG3 polypeptides, which are expected to be suitable for introduction to yeast. Natural variations in the amino acid sequence are not expected to affect its function. Based on BLAST and Clustal W data, it is apparent that the exemplified S. cerevisiae MIG3 polypeptide shares sequence identity to polypeptides from other organisms. Over-expression of functionally and/or structurally similar proteins, homologous proteins and/or substantially similar or identical proteins, is expected to produce similar beneficial results.

In particular embodiments of the present compositions and methods, the amino acid sequence of the MIG3 polypeptide that is over-expressed in modified yeast cells has at least about 50%, at least about 60%, at least about 70%, at least about 80%, at least about 90%, at least about 91%, at least about 92%, at least about 93%, at least about 94%, at least about 95%, at least about 96%, at least about 97%, at least about 98%, or even at least about 99% identity, to SEQ ID NO: 5.

### IV. Modified Yeast Cells Having Increased KGD2 Expression in Combination with an Exogenous PKL Pathway

Increased expression of KGD2, optionally in combination with increased expression of MIG3, can also be combined with expression of genes in the PKL pathway to further increase the production ethanol that is associated with introducing an exogenous PKL pathway into yeast.

Engineered yeast cells having a heterologous PKL pathway have been previously described in WO2015148272 (Miasnikov et al.). These cells express heterologous phosphoketolase (PKL), phosphotransacetylase (PTA) and acetylating acetyl dehydrogenase (AADH), optionally with other enzymes, to channel carbon flux away from the glycerol pathway and toward the synthesis of acetyl-CoA, which is then converted to ethanol. Such modified cells are capable of increased ethanol production in a fermentation process when compared to otherwise-identical parent yeast cells.

### V. Combination of Increased KGD2 Production with Other Mutations that Affect Alcohol Production

In some embodiments, in addition to expressing increased amounts of KGD2 polypeptides, optionally in combination with increased expression of MIG3, and optionally in combination with a heterologous PKL pathway, the present modified yeast cells include additional beneficial modifications.

The modified cells may further include mutations that result in attenuation of the native glycerol biosynthesis pathway, which are known to increase alcohol production. Methods for attenuation of the glycerol biosynthesis pathway in yeast are known and include reduction or elimination of endogenous NAD-dependent glycerol 3-phosphate dehydrogenase (GPD) or glycerol phosphate phosphatase activity (GPP), for example by disruption of one or more of the genes GPD1, GPD2, GPP1 and/or GPP2. See, e.g., U.S. Pat. No. 9,175,270 (Elke et al.), U.S. Pat. No. 8,795,998 (Pronk et al.) and U.S. Pat. No. 8,956,851 (Argyros et al.). Methods to enhance the reuse glycerol pathway by over-expression of glycerol dehydrogenase (GCY1) and dihydroxyacetone kinase (DAK1) to convert glycerol to dihydroxyacetone phosphate (Zhang et al. (2013) J. Ind. Microbiol. Biotechnol. 40:1153-60).

The modified yeast may further feature increased acetyl-CoA synthase (also referred to acetyl-CoA ligase) activity (EC 6.2.1.1) to scavenge (i.e. capture) acetate produced by chemical or enzymatic hydrolysis of acetyl-phosphate (or present in the culture medium of the yeast for any other reason) and converts it to Ac-CoA. This partially reduces the undesirable effect of acetate on the growth of yeast cells and may further contribute to an improvement in alcohol yield. Increasing acetyl-CoA synthase activity may be accomplished by introducing a heterologous acetyl-CoA synthase gene into cells, increasing the expression of an endogenous acetyl-CoA synthase gene and the like.

In some embodiments the modified cells may further include a heterologous gene encoding a protein with NAD⁺-dependent acetylating acetaldehyde dehydrogenase activity and/or a heterologous gene encoding a pyruvate-formate lyase. The introduction of such genes in combination with attenuation of the glycerol pathway is described, e.g., in U.S. Pat. No. 8,795,998 (Pronk et al.). In some embodiments of the present compositions and methods the yeast expressly lacks a heterologous gene(s) encoding an acetylating acetaldehyde dehydrogenase, a pyruvate-formate lyase or both.

In some embodiments, the present modified yeast cells may further over-express a sugar transporter-like (STL1) polypeptide to increase the uptake of glycerol (see, e.g., Ferreira et al. (2005) Mol. Bio. Cell. 16:2068-76; Dus̆ková et al. (2015) Mol. Microbiol. 97:541-59 and WO 2015023989 A1) to increase ethanol production and reduce acetate.

In some embodiments, the present modified yeast cells further include a butanol biosynthetic pathway. In some embodiments, the butanol biosynthetic pathway is an isobutanol biosynthetic pathway. In some embodiments, the isobutanol biosynthetic pathway comprises a polynucleotide encoding a polypeptide that catalyzes a substrate to product conversion selected from the group consisting of: (a) pyruvate to acetolactate; (b) acetolactate to 2,3-dihydroxyisovalerate; (c) 2,3-dihydroxyisovalerate to 2-ketoisovalerate; (d) 2-ketoisovalerate to isobutyraldehyde; and (e) isobutyraldehyde to isobutanol. In some embodiments, the isobutanol biosynthetic pathway comprises polynucleotides encoding polypeptides having acetolactate synthase, keto acid reductoisomerase, dihydroxy acid dehydratase, ketoisovalerate decarboxylase, and alcohol dehydrogenase activity.

In some embodiments, the modified yeast cells comprising a butanol biosynthetic pathway further comprise a modification in a polynucleotide encoding a polypeptide having pyruvate decarboxylase activity. In some embodiments, the yeast cells comprise a deletion, mutation, and/or substitution in an endogenous polynucleotide encoding a polypeptide having pyruvate decarboxylase activity. In some embodiments, the polypeptide having pyruvate decarboxylase activity is selected from the group consisting of: PDC1, PDC5, PDC6, and combinations thereof.

In some embodiments, the yeast cells further comprise a deletion, mutation, over-expression, and/or substitution in one or more endogenous polynucleotides encoding FRA2, ALD6, ADH1, ADH2, GPD2, BDH1, DLS1, DPB3, CPR1, MAL23C, MNN4, PAB1, TMN2, HAC1, PTC1, PTC2, OSM1, GIS1, CRZ1, HUG1, GDS1, CYB2P, SFC1, MVB12, LDB10, C5SD, GIC1, GIC2, JID1 and/or YMR226C.

### VI. Combination of Increased Expression KGD2 with Other Beneficial Mutations

In some embodiments, in addition to increased expression of KGD2 polypeptides, optionally in combination with other genetic modifications that benefit alcohol production, or acetate and/or glycerol reduction, the present modified yeast cells further include any number of additional genes of interest encoding proteins of interest. Additional genes of interest may be introduced before, during, or after genetic manipulations that result in the increased production of active KGD2 polypeptides. Proteins of interest, include selectable markers, carbohydrate-processing enzymes, and other commercially-relevant polypeptides, including but not limited to an enzyme selected from the group consisting of a dehydrogenase, a transketolase, a phosphoketolase, a transaldolase, an epimerase, a phytase, a xylanase, a β-glucanase, a phosphatase, a protease, an α-amylase, a β-amylase, a glucoamylase, a pullulanase, an isoamylase, a cellulase, a trehalase, a lipase, a pectinase, a polyesterase, a cutinase, an oxidase, a transferase, a reductase, a hemicellulase, a mannanase, an esterase, an isomerase, a pectinases, a lactase, a peroxidase and a laccase. Proteins of interest may be secreted, glycosylated, and otherwise-modified.

### VII. Use of the Modified Yeast for Increased Alcohol Production

The present compositions and methods include methods for increasing alcohol production in fermentation reactions. Such methods are not limited to a particular fermentation process. The present engineered yeast is expected to be a “drop-in” replacement for convention yeast in any alcohol fermentation facility. While primarily intended for fuel alcohol production, the present yeast can also be used for the production of potable alcohol, including wine and beer.

### VIII. Yeast Cells Suitable for Modification

Yeasts are unicellular eukaryotic microorganisms classified as members of the fungus kingdom and include organisms from the phyla Ascomycota and Basidiomycota. Yeast that can be used for alcohol production include, but are not limited to, Saccharomyces spp., including S. cerevisiae, as well as Kluyveromyces, Lachancea and Schizosaccharomyces spp. Numerous yeast strains are commercially available, many of which have been selected or genetically engineered for desired characteristics, such as high alcohol production, rapid growth rate, and the like. Some yeasts have been genetically engineered to produce heterologous enzymes, such as glucoamylase or α-amylase.

### IX. Substrates and Products

Alcohol production from a number of carbohydrate substrates, including but not limited to corn starch, sugar cane, cassava, and molasses, is well known, as are innumerable variations and improvements to enzymatic and chemical conditions and mechanical processes. The present compositions and methods are believed to be fully compatible with such substrates and conditions.

Alcohol fermentation products include organic compound having a hydroxyl functional group (—OH) is bound to a carbon atom. Exemplary alcohols include but are not limited to methanol, ethanol, n-propanol, isopropanol, n-butanol, isobutanol, n-pentanol, 2-pentanol, isopentanol, and higher alcohols. The most commonly-made fuel alcohols are ethanol, and butanol.

These and other aspects and embodiments of the present yeast strains and methods will be apparent to the skilled person in view of the present description. The following examples are intended to further illustrate, but not limit, the compositions and methods.

### EXAMPLES

### Example 1

### Materials and Methods

### Liquefact Preparation:

Liquefact (corn mash slurry) was prepared by adding 600 ppm of urea, 0.124 SAPU/g ds acid fungal protease, 0.33 GAU/g ds variant Trichoderma reesei glucoamylase and 1.46 SSCU/g ds Aspergillus kawachii α-amylase, adjusted to a pH of 4.8 with sulfuric acid.

### AnKom Assays:

300 μL of concentrated yeast overnight culture was added to each of a number ANKOM bottles filled with 50 g prepared liquefact (see above) to a final OD of 0.3. The bottles were then incubated at 32° C. with shaking at 150 RPM for 55 hours.

### HPLC Analysis:

Samples of the cultures from AnKom assays were collected in Eppendorf tubes by centrifugation for 12 minutes at 14,000 RPM. The supernatants were filtered using 0.2 μM PTFE filters and then used for HPLC (Agilent Technologies 1200 series) analysis with the following conditions: Bio-Rad Aminex HPX-87H columns, running temperature of 55° C. 0.6 ml/min isocratic flow 0.01 N H₂SO₄, 2.5 μl injection volume. Calibration standards were used for quantification of the of acetate, ethanol, glycerol, glucose and other molecules as needed. All values are reported in g/L.

### RNA-Seq Analysis:

RNA was prepared from individual samples according to the TRIzol method (Life-Tech, Rockville, MD). The RNA was then cleaned up with Qiagen RNeasy Mini Kit (Qiagen, Gennantown, MD). The cDNA from total mRNA in individual samples was generated using Applied Biosystems High Capacity cDNA Reverse Transcription kit (Thermo Fisher Scientific, Wilmington, Delaware). The prepared cDNA of each sample was sequenced using the shotgun method, and then quantified with respect to individual genes. The results are reported as reads per kilobase ten million transcripts (RPK10M) and used to quantify the amount of each transcript in a sample.

### Example 2

### Expression of KGD2, ADR1, DAL80 and MRS6 in Yeast

To understand the regulation of KGD2 (YDR148C) in yeast, RNA-Seq analysis was performed on Saccharomyces cerevisiae strain FERMAX™ Gold (Martrex Inc., Minnesota, USA; herein abbreviated “FG”), a standard strain used for ethanol production. RNA-Seq was performed as described in Example 1. Expression levels are reported as reads per kilobase ten million transcripts (RPK10M).

Overall, the transcription of KGD2 is at low level, and reached highest expression at 24 hr during fermentation. With the objective of choosing a promoter for increasing KGD2 expression late in fermentation, the transcription of ADR1 (YDR216W), DAL80 (YKR034W) and MRS6 (YOR370C) genes was analyzed and compared to KGD2 (Table 1). The transcription of DAL80 reached highest level at 32 hr, then remained almost constant. MRS6 reached highest expression at 36 hr, then declined at 48 hr. Only ADR1 (YDR216VW) gradually increased, and reached its highest level at 48 hr. In order to increase the expression of KGD2 at later stage of fermentation, ADR1 promoter was then selected.

TABLE 1
RNA-Seq analyses of KGD2 and ADR1 (YDR216W) genes
in FG strain during fermentation process

| | Time | | | | | |
|---|---|---|---|---|---|---|
| Gene | 6 h | 12 hr | 24 hr | 30 hrs | 36 hr | 48 hr |
| KGD2 | 926 | 2995 | 3515 | 3397 | 2600 | 1868 |
| ADR1 | 32 | 276 | 2749 | 5957 | 8762 | 15018 |
| DAL80 | 25 | 2459 | 4575 | 8285 | 8284 | 8374 |
| MRS6 | 54 | 1128 | 4486 | 5099 | 7155 | 5893 |

### Example 3

### Preparation of a KGD2 Expression Cassette

The KGD2 gene of Saccharomyces cerevisiae was codon-optimized and synthesized to generate KGD2s. The amino acid sequence of synthesized KGD2s is the same as wild-type KGD2. The ADR1 promoter and CPR1 terminator (YDR155C locus) were functionally linked to the codon-optimized coding sequence of KGD2s to generate the ADR1::KGD2s::CPR1 expression cassette. The KGD2 expression cassette was then introduced downstream of the RPA190 locus (YOR341W) of the FG strain. The expected insertion of the KGD2s expression cassettes in the parental strain was confirmed by PCR.

The amino acid sequence of KGD2 is shown, below, as SEQ ID NO: 1:

MLSRATRTAAAKSLVKSKVARNVMAASFVKRHASTSLFKQANKVESLGSI
YLSGKKISVAANPFSITSNRFKSTSIEVPPMAESLTEGSLKEYTKNVGDF
IKEDELLATIETDKIDIEVNSPVSGTVTKLNFKPEDTVTVGEELAQVEPG
EAPAEGSGESKPEPTEQAEPSQGVAARENSSEETASKKEAAPKKEAAPKK
EVIEPKKADQPKKTVSKAQEPPVASNSFTPFPRTETRVKMNRMRLRIAER
LKESQNTAASLTTFNEVDMSALMEMRKLYKDEIIKKIGTKFGFMGLFSKA
CTLAAKDIPAVNGAIEGDQIVYRDYTDISVAVATPKGLVTPVVRNAESLS
VLDIENEIVRLSHKARDGKLTLEDMTGGTFTISNGGVFGSLYGTPIINSP
QTAVLGLHGVKERPVTVNGQIVSRPMMYLALTYDHRLLDGREAVTFLKTV
KELIEDPRKMLLW

The DNA sequence of the codon-optimized KGD2s coding region is shown, below, as SEQ ID NO: 2:

ATGTTGTCCAGAGCTACCAGAACTGCTGCTGCCAAGTCTTTGGTCAAGTC
CAAAGTTGCCAGAAACGTCATGGCTGCCTCTTTTGTCAAGAGACACGCTT
CTACCAGCTTGTTCAAGCAAGCCAACAAGGTCGAATCCTTAGGTTCTATC
TACTTGTCAGGCAAGAAAATCTCTGTTGCTGCCAATCCATTTTCCATTAC
TTCCAACAGATTCAAGTCTACCAGTATCGAAGTTCCTCCAATGGCCGAAT
CCTTGACCGAAGGTTCTTTGAAGGAATACACCAAGAACGTCGGTGACTTT
ATCAAGGAAGACGAGCTATTAGCTACCATTGAAACCGACAAGATCGATAT
CGAAGTCAACTCTCCAGTTTCTGGTACTGTCACCAAGTTGAACTTCAAGC
CAGAAGACACTGTTACCGTTGGTGAAGAGTTGGCTCAAGTCGAACCAGGT
GAAGCTCCAGCTGAAGGTTCTGGCGAATCCAAGCCAGAACCTACCGAACA
AGCTGAACCATCTCAAGGTGTTGCTGCCAGAGAAAACTCCAGCGAGGAAA
CTGCTTCCAAGAAAGAAGCTGCACCAAAGAAAGAAGCCGCTCCAAAGAAA
GAAGTTACCGAACCCAAAAAGGCTGATCAACCAAAGAAAACCGTCTCCAA
GGCTCAAGAACCTCCAGTTGCTTCCAACTCTTTTACTCCATTTCCACGTA
CCGAAACCAGAGTCAAGATGAACAGAATGAGGTTGAGAATTGCCGAACGT
TTGAAGGAATCTCAAAACACTGCTGCCTCCTTAACTACCTTCAACGAAGT
TGACATGTCTGCTTTGATGGAAATGAGAAAGCTATACAAGGACGAAATTA
TCAAGAAAACTGGTACCAAGTTTGGCTTCATGGGTTTGTTCTCCAAGGCT
TGTACCTTGGCTGCCAAGGACATTCCAGCTGTCAACGGTGCCATCGAAGG
TGACCAAATCGTTTACAGAGATTACACCGACATTTCTGTCGCTGTTGCCA
CTCCAAAGGGTTTGGTCACTCCAGTTGTCAGAAATGCCGAATCCTTGTCT
GTCTTAGACATCGAGAACGAAATTGTTCGTTTGTCTCACAAGGCCAGAGA
TGGCAAGTTAACTCTAGAAGACATGACTGGTGGCACCTTTACTATTTCCA
ATGGTGGCGTCTTTGGTTCCTTGTACGGTACTCCCATTATCAACTCTCCA
CAAACTGCTGTTCTAGGTTTGCACGGTGTCAAGGAAAGACCAGTTACCGT
CAACGGTCAAATCGTTTCCAGACCAATGATGTACTTAGCTTTGACTTACG
ATCACAGATTGCTAGATGGCAGAGAAGCTGTTACCTTCTTGAAGACTGTC
AAGGAATTGATCGAAGATCCAAGAAAGATGTTGCTATGGTAA

The DNA sequence of ADR1 promoter shown, below, as SEQ ID NO: 3:

AAACGCCGGAGGCGACGGCAAGGATGACCATGGCGTCAAGGAACATGCGG
AACATGCTAGAATGATTGCCGAAAGGGCGGGCCCTAGTCAATGGCATCAT
GCGGGAACGTGCAGTAAAGCTCAATGTAACTTGATGCCTATCGGCGTTGT
TCGAGCTAAGGACGATGTGATAGACTGAGGAAGGGTGAACGTCGATAGTT
AGCCGTTGTGAACTAAATAAAAGTGATATTTTTTCGTTGTTCTTTTCTTG
GTTTGGCGGTGTTTTCATTTGTTTTCATGTTTTACTTCGCCACAGGTTTT
CAAAGAACAACGCCTTAAAAATAGGAAAACGTTTTCGCTACAGGTGTTGT
TATTATTGTTGTTGTGCTGTTGTTTATTGTGCTATACTTGTGGTATTTAT
TCTGGACTTCCGATCGGAAATTTTCTTCCCTTGAAGACCTTTTGAAGACA
ACAGTTATATATCATTGATCTGAATTTCTCAGGCTATTTTCAAAATTCCA
TACCTCCTTATTCCAACATTTGCTCGACTACTATAGAAAAGCCTTATTCT
TTTATCTTTGAAAGAAAGAAAAGGTGTCATAGCAAAAGTTTATTGTTACT
CTGTTTTGATATACTCCCTCTTATTCGTTGGAAGTATAAGATTGATTTGC
ATAAATTAACCAATCATTTTGCTACTTTCCCGGTTCTCCCTTTATTATAA
ACACTTCAGAAAAATATTCTGCTACTATTCCTTACTTTACTATAAGAATT
TTGTTTTCCAAAAAAAAAAAATATAAAAAAAATAATCATACTCTATTACT

The DNA sequence of CPR1 terminator region shown, below, as SEQ ID NO: 4:

TTCCGCTCTGCCTGGAACAATACAGCAAAAATTGAAACGAACTATTCTCT
CTTAAATTATATGTATATGTATAAGGTATGTGTATGTATGACAATCAATT
CTTATAACTAACTCTTCCTACCTATATCGGAGTATGTTGCCAGGCTTGCC
AACTAGCCAACTACCGCCTAGGTTATGCCTGTTAAGTTGACGCCGCCGGG
TACCGCCCCCTGTTTTTCCCACGTTTCTTTCTTTCCTCTAGGTAACGGAT
TTTCGGCTTATTTTCCACAGCGAGAAAATTTTCTTTCTCCTGCACTTTTT
TTCATATTCAGGTCCTTTGATCCATTCATTCATTCTTGTTTTAATTTCAA
TCTAGATTTTAGTTTTCGGTTGAATTTATCTTTAGTTTTTTATCAATAAG
AA

### Example 5

### Expression of MIG3 and SU13 in Yeast

To understand the regulation of MIG3 (YER028C) and SUI3 (YPL237W) in yeast, RNA-Seq analysis was performed on strain FG, as above. RNA-Seq was performed as described in Example 1 and the results are summarized in Table 2. Expression levels are expressed as reads per kilobase ten million transcripts (RPK10M). The results indicate that MIG3 was expressed at low levels during fermentation in the FG strain. SU13 was expressed at about 10 times higher than MIG3. The SUI3 promoter was select to drive the expression of MIG3.

TABLE 2
RNA-Seq analysis of MIG expression in FG during fermentation

| | Time | | | | |
|---|---|---|---|---|---|
| Gene | 6 hr | 12 hr | 24 hr | 36 hr | 48 hr |
| MIG3 | 375 | 450 | 561 | 309 | 305 |
| SUI3 | 4075 | 3325 | 2761 | 4144 | 3370 |

### Example 6

### Preparation of a MIG3 Expression Cassette

Saccharomyces cerevisiae MIG3 was codon-optimized and synthesized to generate MIG3s. The amino acid sequence of the synthesized MIG3s is the same as wild-type MIG3. The SU13 promoter and GPD1 terminator were operably linked to the coding-optimized coding sequence of MIG3 to generate the SUI3::MIG3s::GPD1 expression cassette. The MIG3 expression cassette was then introduced downstream of the RPA190 locus (YOR341W) of FG strain. The expected insertion of the MIG3s expression cassettes in the parental strain was confirmed by PCR.

The amino acid sequence of the MIG3 polypeptide is shown, below, as SEQ ID NO: 5:

MNYLRDRFPPDNDQRPFRCEICSRGFHRLEHKKRHVRTHTGEKPHKCTVQ
GCPKSFSRSDELKRHLRTHTKGVQRRRIKSKGSRKTVVNTATAAPTTFNE
NTGVSLTGIGQSKVPPILISVAQNCDDVNIRNTGNNNGIVETQAPAILVP
VINIPNDPHPIPSSLSTTSITSIASVYPSTSPFQYLKSGFPEDPASTPYV
HSSGSSLALGELSSNSSIFSKSRRNLAAMSGPDSLSSSKNQSSASLLSQT
SHPSKSFSRPPTDLSPLRRIMPSVNTGDMEISRIVSVSSSSSSLTSVTYD
DTAAKDMGMGIFFDRPPVTQKACRSNHKYKVNAVSRGRQHERAQFHISGD
DEDSNVHRQESRASNTSPNVSLPPIKSILRQIDNFNSAPSYFSK

The DNA sequence of the codon-optimized MIG3 coding region is shown, below, as SEQ ID NO: 6:

ATGAATTACCTGCGAGATAGATTTCCTCCGGATAATGACCAAAGACCCTT
TAGATGTGAAATTTGTTCACGAGGTTTCCACAGACTTGAACATAAAAAAA
GGCACGTAAGAACGCACACTGGCGAGAAGCCTCACAAATGTACCGTTCAG
GGCTGTCCGAAAAGCTTCAGCCGAAGCGATGAACTAAAAAGACATTTGAG
GACACATACTAAAGGCGTCCAAAGGCGCAGAATAAAATCCAAGGGCTCGC
GAAAAACCGTTGTGAATACTGCTACCGCCGCCCCTACCACCTTCAATGAA
AACACTGGTGTTTCGCTCACGGGGATAGGTCAATCTAAAGTGCCACCTAT
TCTTATCTCCGTTGCTCAGAATTGCGATGACGTGAATATACGAAATACTG
GAAATAATAATGGCATTGTGGAGACACAGGCACCTGCAATTTTAGTGCCT
GTGATAAATATTCCAAATGACCCTCATCCGATTCCAAGTAGCCTCTCCAC
TACTTCTATCACCTCCATTGCATCAGTATATCCCTCTACTTCTCCATTCC
AGTACCTGAAAAGCGGGTTTCCTGAAGATCCTGCATCTACACCGTATGTA
CATTCGTCCGGAAGTTCTTTAGCCCTGGGTGAATTGTCTTCAAACTCCTC
TATATTTTCGAAATCTAGGAGGAATTTGGCCGCCATGAGTGGTCCTGATT
CTTTGAGTAGTTCTAAAAACCAATCCAGTGCTTCGCTTCTTTCTCAAACT
TCACATCCATCAAAGAGCTTTTCAAGACCGCCAACAGACTTAAGTCCTCT
GCGAAGAATCATGCCTTCTGTAAACACAGGAGACATGGAAATTTCAAGGA
CAGTATCCGTTTCGAGCAGTTCATCATCACTCACTTCTGTTACGTATGAT
GACACCGCGGCTAAAGACATGGGCATGGGAATATTTTTTGATAGGCCACC
TGTAACACAGAAAGCTTGCAGGAGCAATCATAAGTACAAGGTTAATGCTG
TTAGCAGAGGGAGACAACATGAAAGGGCACAATTTCATATATCTGGAGAT
GATGAGGACAGTAACGTTCACCGCCAAGAATCAAGAGCATCCAACACAAG
TCCCAATGTATCATTGCCTCCGATAAAGAGCATTTTGCGACAAATTGATA
ATTTCAACAGTGCTCCTTCTTACTTCAGTAAATAA

The DNA sequence of the SUI3 promoter is shown, below, as SEQ ID NO: 7:

CTTTTGTTAATGAGGTGAACAAAACAGGCAACACGGCTTTACATTGGGCG
TCGTTGAATGGCAAATTAGACGTGGTCAAGCTACTGTGTGATGAATATGA
GGCAGACCCCTTTATTAGAAACAAATTCGGCCACGATGCTATCTTTGAGG
CCGAGAACAGCGGGAAGGAAGAAGTGGAAACATACTTTTTGAAGAAGTAT
GATGTCGAACCTGAAGATGATGAAGAAGACACACAAACTGAGGGCAAGAA
TTCGGTCCAAATCACAAAGGGTACAGAAATTGAACAAGTCACCAAGGAAG
CCACCGAGGCTTTAAGAGAAGAAACCGAGAAACTGAATATAAATAAAGAC
TAAAGTAAAGAGCTTGTTTTCTTCGTGGAATAAAAGCCGTAATATCCATT
GAGCATGATATTTTATTTCTTGGTACCGGAGAAGATAAAAGGATGGAACC
AGCAGAAATGCATTAATAAATACAAAAAATTTAGAAAAGAGTTACAGTAA
TAATGTATATTTCTCTCACAAACTAAGTAACCGCTTCAAAAGCGTATATT
TTGAATGCATTGAACTTTCCATTTCATTACCCGCAGAGCCGGAGTCCTCA
TCAACGACGGAGTGAAAAATTTTGTAATTGCGAGAAAAAGTGAAATTGAT
GGAAAAAAAGAAAAAGAAAGTAGAAGAAGACATTATAAGAGAGACTAGGA
AACTTCTTGCACATCAACCGAAAAGCGCCTAGGCAACCAGTCATATAATA
AGCACGCACGAG

The DNA sequence of GPD1 terminator is shown, below, as SEQ ID NO: 8:

ATTTATTGGAGAAAGATAACATATCATACTTTCCCCCACTTTTTTCGAGG
CTCTTCTATATCATATTCATAAATTAGCATTATGTCATTTCTCATAACTA
CTTTATCACGTTAGAAATTACTTATTATTATTAAATTAATACAAAATTTA
GTAACCAAATAAATATAAATAAATATGTATATTTGAATTTTAAAAAAAAA
ATCCTATAGAGCAAAAGGATTTTCCATTATAATATTAGCTGTACACCTCT
TCCGCATTTTTTGAGGGTGGTTACAACACCACTCATTCAGAGGCTGTCGG
CACAGTTGCTTCTAGCATCTGGCGTCCGTATGTATGGGTGTATTTTAAAT
AA

### Example 6

### Generation of an Expression Cassette for Expressing Both KGD2 and MIG3

Construct pZK90-D2G3 contains both the KGD2 and MIG3 over-expression cassettes described in Examples 3 and 6. The DNA fragment containing both KGD2 and MIG3 over-expression cassettes was introduced downstream of the RPA190 locus (YOR341W) of the FG strain. The expected insertion of the KGD2s and MIG3s expression cassettes in the parental strain was confirmed by PCR. Features of the construct are summarized in Table 3.

TABLE 3
Components of the DNA fragment with KGD2
and MIG3 over-expression cassettes

| Gene | Promoter | Terminator |
|---|---|---|
| KGD2 | ADR1 | CPR1 |
| MIG3 | SUI3 | GPD1 |

### Example 7

### Alcohol Production Using Yeast that Over-Express KGD2, MIG3 or KGD2 and MIG3 Together

Yeast strains over-expressing KGD2 (FG-KGD2), MIG3 (FG-MIG3) or KGD2 and MIG3 together (FG-KGD2+MIG3), along with the corresponding parental strain FG were tested by Ankom assay containing 50 g liquefact, as described in Example 1. Fermentations were performed for 32° C. for 55 hr. Samples from the end of fermentation were analyzed by HPLC. The results are summarized in Table 4.

TABLE 4
HPLC results from a small vial assay

| | | | | | Acetate | Ethanol | Glycerol |
|---|---|---|---|---|---|---|---|
| | Glycerol | Acetate | Glucose | Ethanol | reduction | Increase | reduction |
| Strain | (g/L) | (g/L) | (g/L) | (g/L) | (%) | (%) | (%) |
| FG | 11.62 | 1.36 | 0.50 | 137.04 | -0- | -0- | -0- |
| FG-KGD2 | 11.64 | 1.37 | 0.43 | 139.83 | -0- | +2.0 | -0- |
| FG-MIG3 | 10.34 | 1.06 | 1.85 | 137.99 | −28.3 | +0.7 | −12.4 |
| FG- | 10.51 | 1.07 | 1.14 | 140.48 | −27.1 | +2.5 | −10.6 |
| KGD2 + MIG3 | | | | | | | |

Over-expression of KGD2 using the ADR1 promoter resulted in an approximately 2% increase in ethanol production compared to parental strain FG, without appreciably impacting the production of acetate and glycerol. Over-expression of MIG3 using the SUI3 promoter resulted in a decrease in acetate and glycerol production of about 28%, and 12%, respectively.

Surprisingly, over-expression of MIG3 using the SUI3 promoter also resulted in 0.7% increased ethanol production, unlike MIG3 over-expression using the EFB1 promoter (data not shown). Notably, the EFB1 promoter is much stronger than the SUI3 promoter. However, the EFB1 promoter drives peak expression levels very early in fermentation rather than almost constant in fermentation as does the SUI3 promoter. Over-expression of KGD2 using the ADR1 promoter and MIG3 using the SUI3 promoter together resulted in an approximately 2.5% increased ethanol production, and in a decrease in acetate and glycerol production of 27%, and 10.6%, respectively.

What is claimed is:

1. Modified yeast cells derived from parental yeast cells, the modified cells comprising a genetic alteration that causes the modified cells to produce an increased amount of α-ketoglutarate dehydrogenase (KGD2) polypeptides compared to the parental cells, wherein the modified cells produce during fermentation an increased amount of ethanol compared to the amount of ethanol produced by otherwise identical parental yeast cells.

2. The modified cells of claim 1, wherein the genetic alteration comprises the introduction into the parental cells of a nucleic acid capable of directing the expression of a KGD2 polypeptide to a level above that of the parental cell grown under equivalent conditions.

3. The modified cells of claim 2, wherein the genetic alteration comprises the introduction of an expression cassette for expressing a KGD2 polypeptide.

4. The modified cells of claim 3, wherein the expression cassette comprises an exogenous KGD2 gene.

5. The modified cells of claim 2, wherein the nucleic acid comprises a promoter that results in increased expression of KGD2 polypeptides late in fermentation.

6. The modified cells of claim 2, wherein the nucleic acid comprises the ADR1 promoter operably linked to the coding sequence for the KGD2 polypeptide.

7. The modified cells of any of claims 1-6, wherein the amount of increase in the expression of a KGD2 polypeptide is at least 20%, at least 30%, at least 40%, at least 50%, at least 70%, at least 100%, at least 150%, at least 200%, or at least 500% or more, compared to the level expression in the parental cells grown under equivalent conditions.

8. The modified cells of any of claims 1-6, wherein the increase in the amount of KGD2 mRNA produced by the modified cells is at least 2-fold, at least 5-fold, at least 10-fold, at least 20-fold, at least 50-fold, at least 100-fold or more, compared to the amount of KGD2 mRNA produced by the parental cells grown under equivalent conditions.

9. The modified cells of any of claims 1-8, wherein the cells further comprise a genetic alteration that causes the modified cells to produce an increased amount of transcriptional regulator MIG3 polypeptides compared to the parental cells.

10. The modified cells of any of claims 1-9, wherein the cells further comprise an exogenous gene encoding a carbohydrate processing enzyme.

11. The modified cells of any of claims 1-10, further comprising a PKL pathway.

12. The modified cells of any of claims 1-11, further comprising an alteration in the glycerol pathway and/or the acetyl-CoA pathway.

13. The modified cells of any of claims 1-12, further comprising an alternative pathway for making ethanol.

14. The modified cells of any of claims 1-13, wherein the cells are of a Saccharomyces Spp.

15. A method for increased production of alcohol from yeast cells grown on a carbohydrate substrate, comprising: introducing into parental yeast cells a genetic alteration that increases the production of KGD2 polypeptides compared to the amount produced in otherwise identical parental cells.

16. The method of claim 15, wherein the modified cells having the introduced genetic alteration are the modified cells are the cells of any of claims 1-14.

17. The method of claim 15 or 16, wherein the increased production of alcohol is at least 0.5% under equivalent fermentation conditions.

18. The method of any of claims 15-17, wherein the increase in production of KGD2 is an increase of at least 20%, at least 30%, at least 40%, at least 50%, at least 70%, at least 100%, at least 150%, at least 200%, or at least 500% or more compared to the amount of KGD2 produced by otherwise identical parental cells grown under equivalent conditions.

19. The method of any of claims 15-18, wherein the increase in the amount of KGD2 mRNA produced by the modified cells is at least 2-fold, at least 5-fold, at least 10-fold, at least 20-fold, at least 50-fold, at least 100-fold or more compared to the amount of KGD2 mRNA produced by otherwise identical parental cells grown under equivalent conditions.


SEQUENCE LISTING (8 sequences)

SEQ ID NO: 1 - protein, 463 aa, S. cerevisiae
MLSRATRTAAAKSLVKSKVARNVMAASFVKRHASTSLFKQANKVESLGSIYLSGKKISVAANPFSITSNRFKSTSIEVPPMAESLTEGSLKEYTKNVGDFIKEDELLATIETDKIDIEVNSPVSGTVTKLNFKPEDTVTVGEELAQVEPGEAPAEGSGESKPEPTEQAEPSQGVAARENSSEETASKKEAAPKKEAAPKKEVTEPKKADQPKKTVSKAQEPPVASNSFTPFPRTETRVKMNRMRLRIAERLKESQNTAASLTTFNEVDMSALMEMRKLYKDEIIKKTGTKFGFMGLFSKACTLAAKDIPAVNGAIEGDQIVYRDYTDISVAVATPKGLVTPVVRNAESLSVLDIENEIVRLSHKARDGKLTLEDMTGGTFTISNGGVFGSLYGTPIINSPQTAVLGLHGVKERPVTVNGQIVSRPMMYLALTYDHRLLDGREAVTFLKTVKELIEDPRKMLLW

SEQ ID NO: 2 - DNA, 1392 bp, Artificial Sequence
  KGD2
atgttgtccagagctaccagaactgctgctgccaagtctttggtcaagtccaaagttgccagaaacgtcatggctgcctcttttgtcaagagacacgcttctaccagcttgttcaagcaagccaacaaggtcgaatccttaggttctatctacttgtcaggcaagaaaatctctgttgctgccaatccattttccattacttccaacagattcaagtctaccagtatcgaagttcctccaatggccgaatccttgaccgaaggttctttgaaggaatacaccaagaacgtcggtgactttatcaaggaagacgagctattagctaccattgaaaccgacaagatcgatatcgaagtcaactctccagtttctggtactgtcaccaagttgaacttcaagccagaagacactgttaccgttggtgaagagttggctcaagtcgaaccaggtgaagctccagctgaaggttctggcgaatccaagccagaacctaccgaacaagctgaaccatctcaaggtgttgctgccagagaaaactccagcgaggaaactgcttccaagaaagaagctgcaccaaagaaagaagccgctccaaagaaagaagttaccgaacccaaaaaggctgatcaaccaaagaaaaccgtctccaaggctcaagaacctccagttgcttccaactcttttactccatttccacgtaccgaaaccagagtcaagatgaacagaatgaggttgagaattgccgaacgtttgaaggaatctcaaaacactgctgcctccttaactaccttcaacgaagttgacatgtctgctttgatggaaatgagaaagctatacaaggacgaaattatcaagaaaactggtaccaagtttggcttcatgggtttgttctccaaggcttgtaccttggctgccaaggacattccagctgtcaacggtgccatcgaaggtgaccaaatcgtttacagagattacaccgacatttctgtcgctgttgccactccaaagggtttggtcactccagttgtcagaaatgccgaatccttgtctgtcttagacatcgagaacgaaattgttcgtttgtctcacaaggccagagatggcaagttaactctagaagacatgactggtggcacctttactatttccaatggtggcgtctttggttccttgtacggtactcccattatcaactctccacaaactgctgttctaggtttgcacggtgtcaaggaaagaccagttaccgtcaacggtcaaatcgtttccagaccaatgatgtacttagctttgacttacgatcacagattgctagatggcagagaagctgttaccttcttgaagactgtcaaggaattgatcgaagatccaagaaagatgttgctatggtaa

SEQ ID NO: 3 - DNA, 800 bp, Artificial Sequence
  ADR1 Promoter
aaacgccggaggcgacggcaaggatgaccatggcgtcaaggaacatgcggaacatgctagaatgattgccgaaagggcgggccctagtcaatggcatcatgcgggaacgtgcagtaaagctcaatgtaacttgatgcctatcggcgttgttcgagctaaggacgatgtgatagactgaggaagggtgaacgtcgatagttagccgttgtgaactaaataaaagtgatattttttcgttgttcttttcttggtttggcggtgttttcatttgttttcatgttttacttcgccacaggttttcaaagaacaacgccttaaaaataggaaaacgttttcgctacaggtgttgttattattgttgttgtgctgttgtttattgtgctatacttgtggtatttattctggacttccgatcggaaattttcttcccttgaagaccttttgaagacaacagttatatatcattgatctgaatttctcaggctattttcaaaattccatacctccttattccaacatttgctcgactactatagaaaagccttattcttttatctttgaaagaaagaaaaggtgtcatagcaaaagtttattgttactctgttttgatatactccctcttattcgttggaagtataagattgatttgcataaattaaccaatcattttgctactttcccggttctccctttattataaacacttcagaaaaatattctgctactattccttactttactataagaattttgttttccaaaaaaaaaaaatataaaaaaaataatcatactctattact

SEQ ID NO: 4 - DNA, 402 bp, Artificial Sequence
  CPR1 terminator region
ttccgctctgcctggaacaatacagcaaaaattgaaacgaactattctctcttaaattatatgtatatgtataaggtatgtgtatgtatgacaatcaattcttataactaactcttcctacctatatcggagtatgttgccaggcttgccaactagccaactaccgcctaggttatgcctgttaagttgacgccgccgggtaccgccccctgtttttcccacgtttctttctttcctctaggtaacggattttcggcttattttccacagcgagaaaattttctttctcctgcactttttttcatattcaggtcctttgatccattcattcattcttgttttaatttcaatctagattttagttttcggttgaatttatctttagttttttatcaataagaa

SEQ ID NO: 5 - protein, 394 aa, S. cerevisiae
MNYLRDRFPPDNDQRPFRCEICSRGFHRLEHKKRHVRTHTGEKPHKCTVQGCPKSFSRSDELKRHLRTHTKGVQRRRIKSKGSRKTVVNTATAAPTTFNENTGVSLTGIGQSKVPPILISVAQNCDDVNIRNTGNNNGIVETQAPAILVPVINIPNDPHPIPSSLSTTSITSIASVYPSTSPFQYLKSGFPEDPASTPYVHSSGSSLALGELSSNSSIFSKSRRNLAAMSGPDSLSSSKNQSSASLLSQTSHPSKSFSRPPTDLSPLRRIMPSVNTGDMEISRTVSVSSSSSSLTSVTYDDTAAKDMGMGIFFDRPPVTQKACRSNHKYKVNAVSRGRQHERAQFHISGDDEDSNVHRQESRASNTSPNVSLPPIKSILRQIDNFNSAPSYFSK

SEQ ID NO: 6 - DNA, 1185 bp, Artificial Sequence
  MIG3 coding region
atgaattacctgcgagatagatttcctccggataatgaccaaagaccctttagatgtgaaatttgttcacgaggtttccacagacttgaacataaaaaaaggcacgtaagaacgcacactggcgagaagcctcacaaatgtaccgttcagggctgtccgaaaagcttcagccgaagcgatgaactaaaaagacatttgaggacacatactaaaggcgtccaaaggcgcagaataaaatccaagggctcgcgaaaaaccgttgtgaatactgctaccgccgcccctaccaccttcaatgaaaacactggtgtttcgctcacggggataggtcaatctaaagtgccacctattcttatctccgttgctcagaattgcgatgacgtgaatatacgaaatactggaaataataatggcattgtggagacacaggcacctgcaattttagtgcctgtgataaatattccaaatgaccctcatccgattccaagtagcctctccactacttctatcacctccattgcatcagtatatccctctacttctccattccagtacctgaaaagcgggtttcctgaagatcctgcatctacaccgtatgtacattcgtccggaagttctttagccctgggtgaattgtcttcaaactcctctatattttcgaaatctaggaggaatttggccgccatgagtggtcctgattctttgagtagttctaaaaaccaatccagtgcttcgcttctttctcaaacttcacatccatcaaagagcttttcaagaccgccaacagacttaagtcctctgcgaagaatcatgccttctgtaaacacaggagacatggaaatttcaaggacagtatccgtttcgagcagttcatcatcactcacttctgttacgtatgatgacaccgcggctaaagacatgggcatgggaatattttttgataggccacctgtaacacagaaagcttgcaggagcaatcataagtacaaggttaatgctgttagcagagggagacaacatgaaagggcacaatttcatatatctggagatgatgaggacagtaacgttcaccgccaagaatcaagagcatccaacacaagtcccaatgtatcattgcctccgataaagagcattttgcgacaaattgataatttcaacagtgctccttcttacttcagtaaataa

SEQ ID NO: 7 - DNA, 762 bp, Artificial Sequence
  SUI3 promoter
cttttgttaatgaggtgaacaaaacaggcaacacggctttacattgggcgtcgttgaatggcaaattagacgtggtcaagctactgtgtgatgaatatgaggcagacccctttattagaaacaaattcggccacgatgctatctttgaggccgagaacagcgggaaggaagaagtggaaacatactttttgaagaagtatgatgtcgaacctgaagatgatgaagaagacacacaaactgagggcaagaattcggtccaaatcacaaagggtacagaaattgaacaagtcaccaaggaagccaccgaggctttaagagaagaaaccgagaaactgaatataaataaagactaaagtaaagagcttgttttcttcgtggaataaaagccgtaatatccattgagcatgatattttatttcttggtaccggagaagataaaaggatggaaccagcagaaatgcattaataaatacaaaaaatttagaaaagagttacagtaataatgtatatttctctcacaaactaagtaaccgcttcaaaagcgtatattttgaatgcattgaactttccatttcattacccgcagagccggagtcctcatcaacgacggagtgaaaaattttgtaattgcgagaaaaagtgaaattgatggaaaaaaagaaaaagaaagtagaagaagacattataagagagactaggaaacttcttgcacatcaaccgaaaagcgcctaggcaaccagtcatataataagcacgcacgag

SEQ ID NO: 8 - DNA, 352 bp, Artificial Sequence
  GPD1 terminator
atttattggagaaagataacatatcatactttcccccacttttttcgaggctcttctatatcatattcataaattagcattatgtcatttctcataactactttatcacgttagaaattacttattattattaaattaatacaaaatttagtaaccaaataaatataaataaatatgtatatttgaattttaaaaaaaaaatcctatagagcaaaaggattttccattataatattagctgtacacctcttccgcattttttgagggtggttacaacaccactcattcagaggctgtcggcacagttgcttctagcatctggcgtccgtatgtatgggtgtattttaaataa